(19) United States
(12) Patent Application Publication
(10) Pub. No.: US 2003/0199784 A1
(43) Pub. Date: Oct. 23, 2003

(54) Non-invasive cerebral spinal fluid pressure monitor apparatus and method

(76) Inventor: Martin L. Lenhardt, Hayes, VA (US)

Correspondence Address: Phillip J. Articola, FOLEY & LARDNER, Washington Harbour, 3000 K Street, N.W., Suite 500, Washington, DC, 20007-5109, US

(21) Appl. No.: 09/973,992

(22) Filed: Oct. 11, 2001

Related U.S. Application Data
(60) Provisional application No. 60/253,066, filed on Nov. 27, 2000.

(51) Int. Cl. A61B 5/00

(52) U.S. Cl. 600/561

(57) ABSTRACT

A non-invasive cerebral spinal fluid pressure method and apparatus applies at least one signal to a head of a subject, where that at least one signal is provided at a frequency range that includes the brain resonant frequency. The received signal strength is an indication of the amount of cerebral spinal fluid pressure in the head of the subject.

### BACKGROUND OF THE INVENTION

1. Field of the Invention

The present invention relates to a cerebral spinal fluid (CSF) pressure monitor apparatus and method, and in particular, to a non-invasive way of determining the CSF pressure in a person's skull.

2. Description of the Related Art

Accurately determining the CSF pressure in a person's skull is important especially when that person has been recently involved in a serious accident or other type of accident that has resulted in trauma to that person's head, particularly to the brain. The CSF pressure provides an indication as to whether or not the accident victim needs to be given immediate medical treatment at the site of the accident (e.g., tapping a hole into the skull to relieve the CSF pressure in the skull), or whether that person can be stabilized in a less extreme manner and then transported to a nearest hospital for more extensive treatment in an emergency room. Furthermore, while an accident victim is being transported in an ambulance, there is a desire to send current CSF pressure information to the hospital, so that a doctor who will be working on the accident victim will have up-to-date information that will assist him/her in treating the accident victim correctly as soon as that person arrives at the hospital.

CSF pressure can be measured in a direct manner by performing a spinal tap (lumbar puncture) to the spine of a person, and thereby measure the CSF pressure there. There is a direct relationship between the CSF pressure measured at the spine and the CSF pressure around the person's brain. However, a spinal tap is an invasive procedure that has some risks associated with it, such as the risk of bacteria getting into the spinal column of the patient due to relative slow healing time. This could lead to serious complications for the patient.

Therefore, non-invasive CSF pressure monitors are highly desired, which do not have risks associated with determining CSF pressure by invasive techniques.

U.S. Pat. No. 4,186,741, issued to Fleischmann, relates to a non-invasive pressure sensor apparatus for communicating pressure inside a body to the exterior of the body. Fleischmann's apparatus using a bellows contained within a housing, and the apparatus is fully implantable on the patient's head. The pressure in the person's skull causes the bellows to expand and contract linearly as a function of the body pressure, and the linear movement of the bellows is communicated to a receiver located external to the body to thereby provide data indicative of the pressure.

U.S. Pat. No. 4,204,547, issued to Allocca, relates to a non-invasive intracranial pressure monitor that occludes the jugular vein at a selected location in order to interrupt the flow of blood temporarily. Then, the rate of blood flow within the jugular is determined upstream of the selected location over a predetermined period of time following the occlusion, in order to obtain an estimate of the CSF pressure.

U.S. Pat. No. 4,564,022, issued to Rosenfeld et al., relates to a non-invasive device that estimates the intercranial pressure by a measurement of visual evoke potentials, electric brain waves which are caused by visual stimulation, with the use of an electroencephalogram signal. The peak of the second negative-going wave (N2 wave) is identified, and its latency measured. The subject's intracranial pressure is then estimated by comparing the value of the latency with a known latency/intracranial pressure correlation.

U.S. Pat. No. 4,984,567, issued to Kageyama et al., relates to a non-invasive intracranial pressure monitor that utilizes an ultrasonic pulse to measure the thickness of the dura mater, which is a thick membrane that envelops the brain, and the change thereof. According to Kageyama et al., the intracranial pressure and its change can be calculated by utilizing the correlation between the intracranial pressure and the dura matter thickness.

U.S. Pat. Nos. 5,074,310 and 5,117,835, both issued to Mick, relate to measuring the intracranial pressure by generating a predetermined vibration signal which is applied to a location on the patient's skull, detecting an output vibration from another location on the skull, storing data characteristic of the two signals, repeating these steps over time, and then analyzing the data to diagnose changes in ICP over time. FIG. 5 of each of these references shows that a relationship between intra skull pressure and harmonic frequency shift in skull resonance, where CSF pressure increases with frequency, and where the harmonic frequency of the skull is in a range of from 300 Hz to 700 Hz, based on the particular CSF pressure currently existing in the skull.

U.S. Pat. No. 5,388,583, issued to Ragauskas et al., relates to non-invasively deriving an indication of dynamic characteristics of cerebrovascular and intracranial blood pressure activity from a measurement of travel times of ultrasonic pulses through the intracranial medium. The basis for the derivation is that the inventors of this patent discovered that when the brain volume decreases, the changes in the acoustic velocity of ultrasonic pulses is directly proportional to the incremental change in intracranium pressure.

U.S. Pat. No. 5,617,873, issued to Yost et al., relates to accurately calibrating non-invasive intracranial monitoring devices by providing known changes in the intracranial pressure by utilizing non-invasive devices, such as a tilting bed and a pressurized skull cap.

Stevanovic et al., in an article entitled “The Effect of Elevated Intracranial Pressure on the Vibrational Response of the Ovine Head”, published in Annals of Biomedical Engineering, 1995, pages 720-727, studied the relationship between CSF pressure and changes in the impedance of a sheep head correlated with frequency of vibration. Stevanovic et al. found that impedance changes were reliably correlated with changes in CSF pressure.

However, the method of Stevanovic et al. involved cutting and retraction of the skin on top of the skull, drilling holes, and direct attachment of an actuator and sensors to the top of the skull with screws. Needless to say, this is a fairly invasive procedure. In that regard, it is likely that Stevanovic et al. was measuring both skull and brain resonances.

Semmlow et al., in an article entitled “A Noninvasive Approach To Intracranial Pressure Monitoring”, Journal of Clinical Engineering, Jan-Mar., 1982, describes an experiment that measured the acoustic properties of the skull, in animals, under normal and elevated intracranial pressures. The results obtained showed that intracranial pressure strongly influences the acoustic response, and at least one feature of the response, that being the damping factor, is related to intracranial pressure in a consistent manner.

Freeman et al., in an article entitled “Bone conduction experiments in animals—evidence for a non-osseous mechanism”, Hearing Research 146, pages 72-80, Apr. 28, 2000, describes auditory nerve-brainstem evoked response (ABR) that is elicited not only with vibration on bone, but also with the vibrator directly on the brain. These results were obtained based on experiments performed on rats and guinea pigs, and a postulation was made that ‘classical’ bone conduction mechanisms should be modified to include a major pathway for cochlear excitation which is non-osseous, where bone vibrations may induce audio-frequency sound pressures in the skull contents which are communicated to the fluids of the inner ear.

While most of the above devices and methods are somewhat useful in estimating CSF pressure in the skull of a patient, there is a need to provide an accurate estimate of the CSF pressure in a patient's skull, using non-invasive techniques.

### SUMMARY OF THE INVENTION

The present invention involves a non-invasive apparatus and/or method for measuring the CSF pressure in a person's skull, by providing pulses to one location at the exterior of the skull and by measuring pulses at another location on the exterior of the skull opposite the one location. By using an appropriate range of frequencies that are frequencies corresponding to brain resonance, an amplitude of the received pulse is obtained, where that amplitude corresponds to a current CSF pressure in the patient's skull.

One aspect of the present invention relates to a method of estimating an intracranial pressure within a head of a subject. That method includes providing a signal to the head of the subject. The method also includes measuring an amplitude of a received signal at a location on the head of the subject different from where the signal was provided to the head of the subject. The method further includes determining the intracranial pressure based on the amplitude of the received signal.

Another aspect of the present invention relates to a method of estimating an intracranial pressure within a head of a subject. That method includes providing a signal to the head of the subject at at least two different times and at at least two different frequency values, respectively, that correspond to a brain resonant frequency and a harmonic of the brain resonant frequency. The method also includes measuring an amplitude of a received signal at a location on the head of the subject different from where the signal was provided to the head of the subject, the measuring being performed at the first and second times. The method further includes determining the intracranial pressure based on the amplitude of the received signal at the first and second times.

Yet another aspect of the present invention relates to an apparatus for estimating an intracranial pressure within a head of a subject. That apparatus includes an adjustable headband that includes at least one transducer and one sensor, the at least one transducer providing a signal to the head of the subject.

The apparatus also includes a computation unit that estimates the intracranial pressure and that provides a signal to the at least one transducer to output the signal. An amplitude of a received signal is measured by the at least one sensor at a location on the head of the subject different from where the signal was provided to the head of the subject. The intracranial pressure is calculated based on the amplitude of the received signal as provided to the computation unit by the at least one sensor.

### BRIEF DESCRIPTION OF THE DRAWINGS

The invention will become more fully apparent from the following detailed description when read in conjunction with the accompanying drawings with like reference numerals indicating corresponding parts throughout, and wherein:

FIG. 1 shows one possible configuration of a headband and elements provided thereon, which can be used as a non-invasive CSF monitoring device according to the present invention;

FIG. 2 shows a configuration of a CSF monitoring system, according to a first embodiment of the invention;

FIG. 3 shows a configuration of a CSF monitoring system, according to a second embodiment of the invention;

FIG. 4A is a plot of received signals at a right ear sensor due to signals provided to the right ear, in both an audible band and a lower-ultrasound band;

FIG. 4B is a plot of received signals at a left ear sensor due to signals provided to the right ear, in both an audible band and a lower ultrasound band;

FIG. 5 is a comparison of brain resonant frequency and harmonics of a sphere model of a brain, a closed tube model of a brain, and an empirical model of the system according to the invention;

FIG. 6 is a plot showing different head resonance frequencies in the audible band up through the lower-ultrasound band;

FIG. 7A is an FFT plot of a back-to-front signal measurement in accordance with an aspect of the invention;

FIG. 7B is an FFT plot of a side-to-side signal measurement in accordance with an aspect of the invention;

FIG. 8 is a plot showing the different brain resonance frequencies obtained by measuring the brain resonance at different points external to the skull;

FIG. 9 shows two different sized brain models and the plot of received signal strength over a range of frequencies of from 10 kHz to 17 kHz, for both a non-pressure condition and a pressure condition;

FIG. 10 is a plot comparing the results obtained by three different brain models, for brain resonance versus brain size;

FIG. 11 shows a one-dimensional ICP model that is used to determine ICP, to obtain a better understanding of how the present invention works;

FIG. 12 is a plot of six different curves, three for baselines and three for valsalvas, for a first subject to show the received signal strength at a lower audio frequency, in accordance with an aspect of the present invention;

FIG. 13 is a plot of six different curves, three for baselines and three for valsalvas, for a second subject to show the received signal strength at a lower audio frequency, in accordance with an aspect of the present invention;

FIG. 14 is a plot of six different curves, three for baselines and three for valsalvas, for a subject to show the received signal strength at an upper audible frequency, in accordance with an aspect of the present invention;

FIG. 15A is a plot of four baselines and FIG. 15B is a plot of three valsalvas for several subjects, to demonstrate that the received signal strength varies with change in CSF pressure, in accordance with an aspect of the present invention;

FIG. 16 is a plot of four different curves, two for baselines and two for valsalvas, for a subject to show the received signal strength at a lower-ultrasound frequency, in accordance with an aspect of the present invention;

FIG. 17 is a plot showing changes in received signal strength over a 15 second period for a valsalva and for a baseline measurement, in accordance with an aspect of the present invention;

FIG. 18 shows a dry/wet skull setup that was used to demonstrate a principle of the present invention;

FIG. 19 is a plot of a received signal strength over a range of frequencies for both a low-pressure condition and a high pressure condition, to demonstrate a principle of the present invention; and

FIG. 20 is a plot of different subjects and their respective standard deviation values of received signal strength obtained over a range of frequencies, in accordance with an aspect of the present invention.

### DETAILED DESCRIPTION OF THE PREFERRED EMBODIMENTS

Preferred embodiments of the invention will be described in detail hereinbelow, with reference to the drawings.

The present invention provides a non-invasive apparatus and/or method for determining the CSF pressure in a person's skull, by using acoustic and higher-frequency signals applied to the skull from the exterior of the skull. Based on measurements of the amplitude of signals that pass through the skull and that are received at another location external to the skull, an estimate of the CSF pressure in the patient's skull can be reliably estimated.

In the present invention, brain resonance is measured under varying degrees of pressure. The above-discussed U.S. patents issued to Mick, on the other hand, measure changes in skull resonances with pressure. The skull is not completely rigid and has some compliance. Under the stress of increased brain pressure, the increased stiffness of the skull changes the resonant frequency upward, as seen in FIG. 5 of each of the Mick patents. As such, Mick measures the changed resonant frequency in order to obtain an estimate of the CSF pressure in the skull.

On the other hand, the present invention measures brain resonance, which does not change in frequency with increased (or decreased) pressure in the skull. What does change, and which is measured in the present invention, is the spread of frequencies (up and down) due to increased damping when the brain is under pressure. The present invention measures the change in the spread of frequencies to estimate the CSF pressure in the skull (and hence the degree of damage to a person's brain due to an accident suffered by the person).

While Mick uses a relatively low frequency signal (300 Hz to 700 Hz) to measure skull resonance and changes thereof, the present invention uses a higher frequency signal (kHz range) to measure the brain resonance. A typical sized and shaped human brain resonates at around 12.5 kHz, and varies somewhat from person to person depending upon brain size and shape. The present invention measures the brain resonant frequency, and frequencies below and above the brain resonant frequency, in order to obtain measurements at harmonics of the brain resonance as well as including frequencies below the brain resonant frequency that correspond to resonance at the brain/skull continuum.

Also, the present invention is different from the system of Stevanovic et al. in that the present invention is completely non-invasive, whereby transducers and sensors are coupled to the exterior of the skin, and whereby the present invention employs a head-through mode of transmission (front-to-back, and/or side-to-side), in contrast to the impedance method of Stevanovic et al. Also, the present invention employs a wider range of frequencies than what is used by Stevanovic et al., thereby decoupling the brain resonances (to be measured) from skull resonances by the choice of appropriate harmonics, and by processing of the signals from the sensors.

The present invention utilizes an actuator and multi-sensor array that makes use of the geometry and resonances of the head to decouple the brain from the skull, and to thereby measure changes due to damping by increased CSF pressure. The array is preferably mounted on a headband, such that received signals from various locations on the head can be processed, using phase differences to cancel skull resonances and enhance brain resonances. The headband is adjustable to fit on any sized head, thereby allowing for precise control of positioning of the array and coupling of the transducers to the skin.

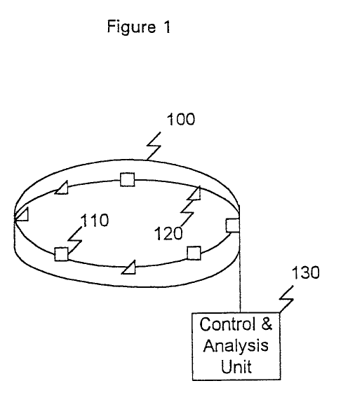

FIG. 1 shows an example of a headband 100 that can be used in the present invention. The headband 100 is preferably expandable and contractible (e.g., elastic headband) to fit any size head. Attached to the headband are a plurality of transducers 110 (shown by square icons in FIG. 1) and a plurality of oppositely-positioned sensors 120 (shown by triangle icons in FIG. 1). The outputs of the sensors 120 are provided to a control and analysis unit 130, whereby that unit 130 also provides control signals to the transducers 110 to have them output pulses at particular instants in time (and at particular frequencies). The connection of the control and analysis unit 130 to the elements on the headband 100 may be either a wired or a wireless connection.

In the present invention, CSF pressure changes from brain resonances are determined based on 1) selection of sensors to enhance brain resonances and discriminate against skull resonances, and 2) use of frequencies ranging from the audible range to the ultrasonic range, with measurement of means and variability at specific resonances. The present invention preferably employs digital filtering, spectrum analysis, and calculation, in determining a CSF pressure.

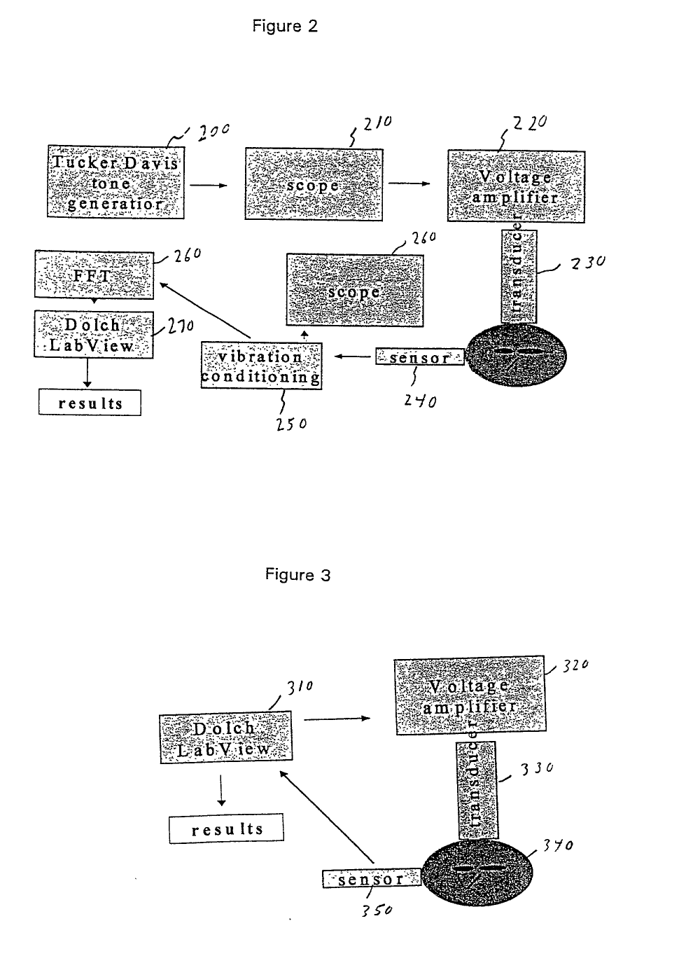

FIG. 2 shows a first embodiment of a system to measure CSF pressure according to the invention. The first embodiment is a computer-enabled system. In FIG. 1, a tone generator 200 is used to generate tones to be provided to a patient's head. An oscilloscope 210 is used to view the tones generated by the tone generator 200. A voltage amplifier 220 is provided to amplify the tones, as needed. A transducer 230 provides the tones to the patient's head, as a vibration. A sensor 240 measures a signal obtained from the tone applied to the patient's head. The output of the sensor 240 is provided to a vibration conditioning circuit 250 and also to an oscilloscope 260 to view the output. The output of the vibration conditioning circuit 250 is provided to an FFT analyzer 260, in order to provide a frequency output to a computer 270 (Flex Pac Lab View was the software used to analyze the results in this configuration; of course, other types of analysis software may be used instead), whereby an analysis of the frequency content was provided in order to obtain an estimate of the CSF pressure in the head.

Another configuration, as a second embodiment of the invention, is shown in FIG. 3. In FIG. 3, a computer (a Dolch computer and Lab View software were used in this configuration; of course, other brands of computers and types of analysis software may be used alternatively) 310 provides a signal to a voltage amplifier 320, which amplifies the signal and provides it to a transducer 330. The transducer 330 is attached to a person's head 340, and a sensor 350 receives a signal from another location on the person's head 340. The output of the sensor 350 is provided to the computer 310 for analysis of the sensor outputs, and results of the analysis (estimated CSF pressure) are output by the computer 310.

The sensors 350 may be any form of vibration pick-up device, including but not limited to a) piezoceramic bimorphs, b) piezoelectric film (PVFT), c) high frequency miniature hydrophone inserted into the ear canal, or d) accelerometer placed on various sites on the skin of a person's neck. The PVFT sensor is disposable and cheap, which is advantageous. The advantage of the hydrophone is that it can always be positioned at the same location (within the ear), but may require some sort of headband for clinical use.

Tests were performed on a subject to determine the CSF pressure in the subject's head using the present invention when the subject was upright, and when the subject was inverted (by the use of a flip recliner). This was done under the assumption that the CSF increases in a person's head when the person is inverted. Signals were provided to the subject's head to determine whether the increased pressure condition resulted in a change in received signal amplitude for frequencies around the brain's resonant frequency. The results were that the received signals did change in amplitude but not in frequency when the subject was inverted, where signals were input to the patient's head within a range of from 4 kHz to 28 kHz. The changes were especially large (4-6 db difference or more) at the brain resonance frequency and at harmonics of the brain resonance frequency.

In order to obtain some validation of the results obtained, those results were compared to other brain models. Under one model (Lim et al.), the brain resonance is modeled to be a function of the speed of sound divided by the brain's diameter times π multiplied by a constant (boundary condition). Modeling the conductive pathway through the brain in one dimension is equivalent to a closed tube. Resonance of the closed tube is a product of the speed of sound divided by two times the length.

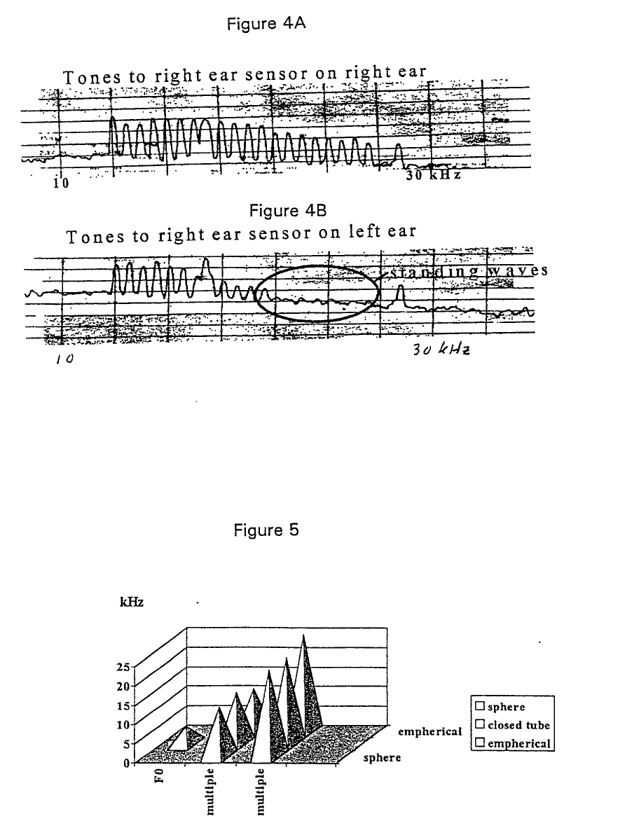

FIG. 4A shows tones received by a right ear sensor based on signals provided to the right ear, and FIG. 4B shows tones received by the right ear sensor based on signals provided to the left ear, for one subject who was in a normal, non-inverted state. The frequency range of the tones was between 10 kHz and 30 kHz. The difference between the plots shown in FIGS. 4A and 4B is most likely due to standing waves that attenuate the signals from 19 kHz to about 26 kHz in FIG. 4B. Note that the resonance peaks are practically the same in FIGS. 4A and 4B.

Resonance is also associated by standing waves in the head when the stimulus wavelength is equal to the brain diameter or some multiple thereof. Under these conditions, the wave would be canceled on one side of the head and would be present at the source side. Perceptually, this will result in a lateralized sound image to one side or the other. This effect is seen by the flat spectrum between about 19 kHz and 27 kHz in FIG. 4B. Subjects tested to verify the method and apparatus of the present invention reported that some tones in the sequence “jumped” across their heads, suggesting traveling waves. The effect is frequency dependent and is most likely related to skull/brain geometry. When standing waves occur, little energy should be present at one side of the head in a specific frequency range, and this phenomenon is shown in FIG. 4B for tones received at another location on the head (which is preferably but not necessarily opposite from where a location where the tones were provided to the head).

Standing waves, that is, incoming waves canceling reflected waves, were measured by modifying the cerebral spinal fluid pressure analysis system of the present invention. For obtaining the plots shown in FIGS. 4A and 4B, an accelerometer was placed on the driving side (right side) of the head and a second accelerometer was placed on the opposite side (left side). Tones were presented in a series and recorded from both sides. Resonance would be expected in the 11 kHz and 22 kHz ranges (brain resonant frequency and first harmonic thereof). The frequencies used were selected to encompass the expected second harmonic of the brain resonance at around 22 kHz.

Data from six subjects were pooled and summarized, and the results are provided in FIG. 5 (as the “empirical” plot). The results obtained confirms the underlying concept of the invention in that brain resonance can be determined based on measurement of signal amplitude for signals provided to the skull, and that increases in CSF pressure will alter the vibrational behavior of the brain. The empirical results obtained from the present invention favorably compare with the closed tube model and the sphere model of the brain.

The present invention is a non-invasive monitor of intracranial pressure that has its primary use for medical applications, especially medical emergencies. The present invention provides an indirect approach that measures vibration analysis within a frequency range that includes the brain resonant frequency (and harmonics thereof) to estimate intracranial pressure changes.

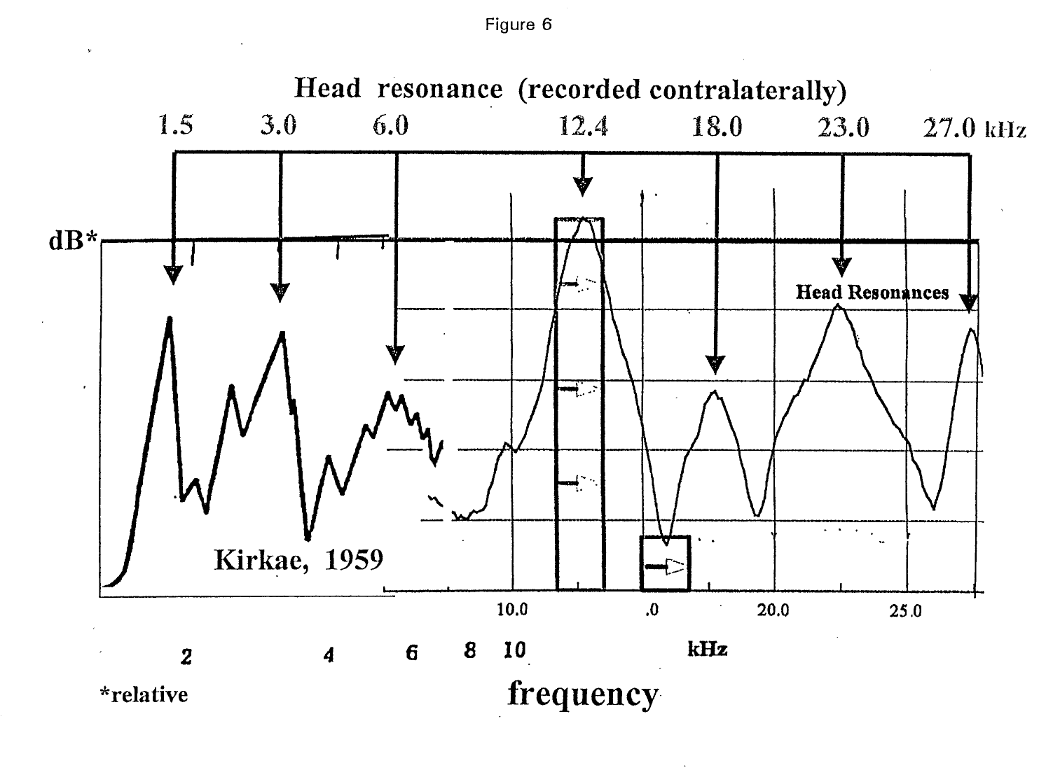

FIG. 6 shows a plot of head resonance, recorded contralaterally. The plot for the lower frequency range of the frequency range shown in FIG. 6 was obtained from a figure provided in an article by Kirkae (1959). FIG. 6 shows head resonances occur at around 1.5 kHz, 3.0 kHz, 6.0 kHz, 12.4 kHz, 18.0 kHz, 23.0 kHz, and 27.0 kHz. The 12.4 kHz resonance is the brain resonant frequency, and the higher resonances are due to harmonics of that resonant frequency. The lower resonances are most likely due to the skull/brain continuum, and measure the skull/brain-coupled resonance. In the preferred embodiment of the present invention, a swept frequency signal is provided to the head, where the sweep is across a range of frequencies that include the audio band and the lower-ultrasonic band, in order to measure received signal strength at each of the different head resonant frequencies. Based on the received information, an accurate measurement of the CSF pressure in the head can be obtained.

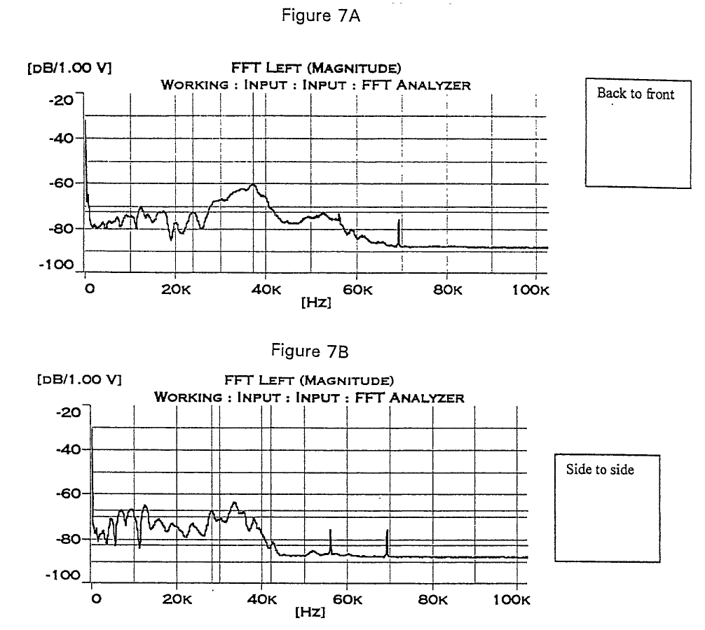

FIG. 7A shows an FFT plot for a back-to-front signal measurement with respect to a subject's head, and FIG. 7B shows an FFT plot for a side-to-side signal measurement of the subject's head. These two figures show that the frequencies of interest are between about 2 kHz to about 36 kHz for measuring brain resonance effects.

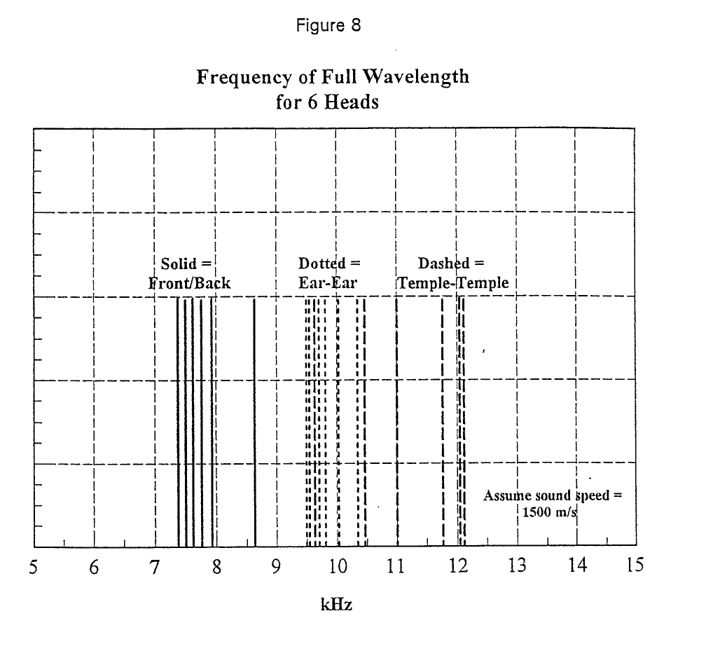

FIG. 8 shows the results obtained from six different persons for determining the fundamental brain resonance for each of those persons, as measured at a different pair of locations (preferably opposite, but not mandatory) on a person's head. The ear-to-ear (lower side-to-side) measurements result in a measured brain resonance at around 10 kHz, the temple-to-temple measurements (upper side-to-side) result in a measured brain resonance at around 12.0 kHz, and the front-to-back measurements result in a measured brain resonance at around 7.75 kHz. These differences are most likely due to the brain not being a perfect sphere, and so the brain resonance frequency is location-dependent. For example, the back-to-front resonance frequency is lower than the others, since the propagation distance is longer and since the brain is not a perfect sphere.

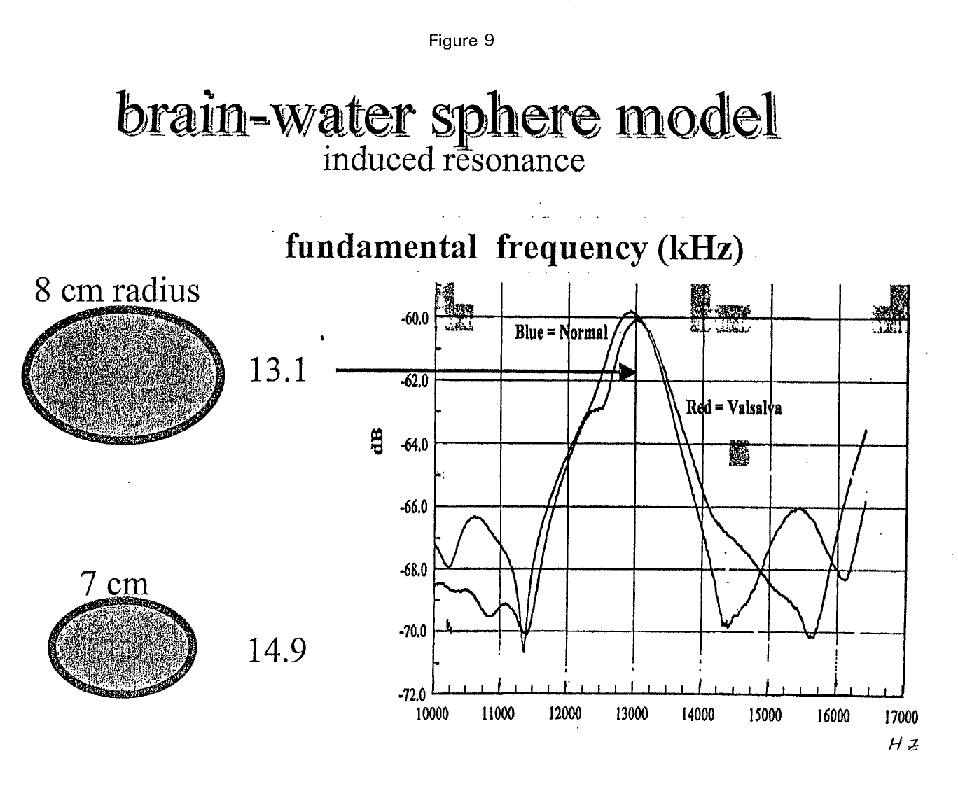

FIG. 9 shows the results obtained from an 8 cm radius brain model, and from a 7 cm radius brain model. In these tests, the brain was modeled as a water balloon placed inside a real human skull. Pressure was applied to the brain model by pressing on the water balloon, and signals were applied to the brain model at normal (no pressure) conditions and at pressure conditions. As seen in the plot in FIG. 9, the received signal strength at the brain resonance frequency (13 kHz) is practically the same whether or not the brain is under pressure. However, at a resonance frequency of about 15.5 kHz, the brain-under-pressure results in a lower received signal, which is about 4 dB lower than is obtained at that same frequency (15.5 kHz) when the brain model was not subjected to pressure.

The present invention utilizes this feature of the determination of the received signal amplitude level in order to determine whether the brain is under pressure, and if so, by how much and whether or not the brain pressure is increasing or decreasing over time. By taking measurements at a range of frequencies above and below the brain resonant frequency (to cover at least one brain resonance harmonic frequency, preferably), an accurate CSF pressure estimated can be obtained. This can be done, for example, by averaging (taking the mean value) the received signal amplitude values at the two different resonance frequencies, and comparing the averaged amplitude to an array of pre-stored averaged amplitude values that indicate a relative CSF pressure. The pre-stored values can be obtained beforehand by running tests on a sample of persons, to get reliable CSF pressure vs. harmonic frequency amplitude values. After these tests are performed, data is stored in a memory that is accessible by the present invention.

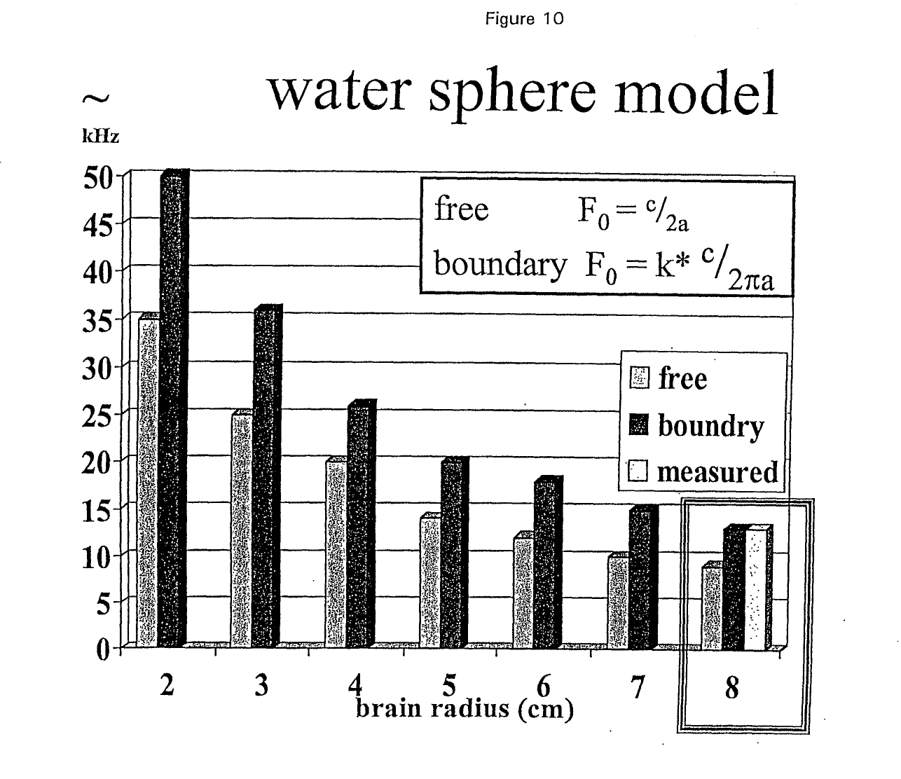

FIG. 10 shows plots for different-sized brains for the water-sphere brain model, with comparison made to different modeling approaches (free, boundary, measured). The measured approach at 8 cm brain size using the present invention matches up well with the idealized (free and boundary) brain resonant model.

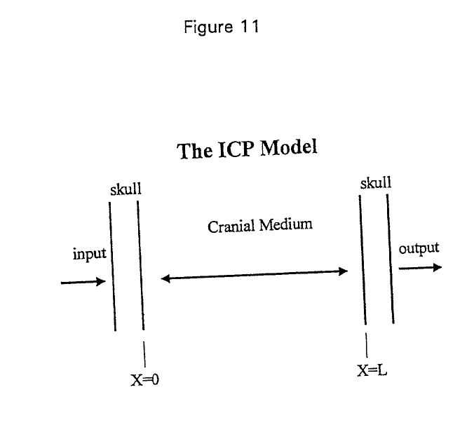

FIG. 11 shows a one-dimensional model of a brain, and is provided to explain the general features of the ICP measurement system of the present invention. By coupling a transducer to provide an audio or ultrasound signal to the skull of a person, the signal is transmitted into the cranial matter. At frequencies of several kHz and higher, the skull highly attenuates the signal provided to it, and therefore, any signal traveling around the skull can be neglected relative to the signal traveling through the cranial matter.

As shown in FIG. 11, the signal enters the material at x=0 and is detected at x=L, where L is the linear dimension of the head. General boundary and propagation equations for the material displacement psi are assumed. The coupling of the signal to the medium is set by the coupling coefficient gamma. The input signal is assumed to be a sinusoid. The cranial material elasticity and attenuation terms are included in the wave equation.

x :=L

The homogeneous solution attenuates rapidly and can be neglected. The inhomogeneous solution results in a pressure wave propagating to the right (in the figure) and a reflected wave propagating to the left. Each of these waves is spatially attenuated and shifted in phase relative to the input signal.

Of particular interest in understanding the ICP measurement system is the measure of the energy detected at L. For example the expression for the time average of the displacement squared is presented.

(a²+b²):=

This energy expression is the product of three terms. First, it is proportional to the magnitude of the input signal. The second term represents a series of resonant terms spaced in frequency at about df=c/(2*L). The depth of the resonant terms depends on the attenuation through beta*L/c. The third term represents a natural spectral transmission region that depends most strongly on the coupling coefficient gamma.

The ICP measurement system transmits energy within the natural spectral transmission region of the head. By examining the depth of the resonant terms a direct measure of the attenuation coefficient. Since beta is proportional to the ICP this provides a direct measure of ICP.

The homogeneous solution attenuates rapidly and can be neglected. The inhomogeneous solution results in a pressure wave propagating to the right (in the figure) and a reflected wave propagating to the left. Each of thee waves is spatially attenuated and shifted in phase relative to the input signal.

Of particular interest in understanding the ICP measurement system is the measure of the energy detected at L. For example the expression for the time average of the displacement squared is presented.

(a²+b²):=

This energy expression is the product of three terms. First, it is proportional to the magnitude of the input signal. The second term represents a series of resonant terms spaced in frequency at about df=c/(2*L). The depth of the resonant terms depends on the attenuation through beta*L/c. The third term represents a natural spectral transmission region that depends most strongly on the coupling coefficient gamma.

The ICP measurement system transmits energy within the natural spectral transmission region of the head. By examining the depth of the resonant terms a direct measure of the attenuation coefficient. Since beta is proportional to the ICP this provides a direct measure of ICP.

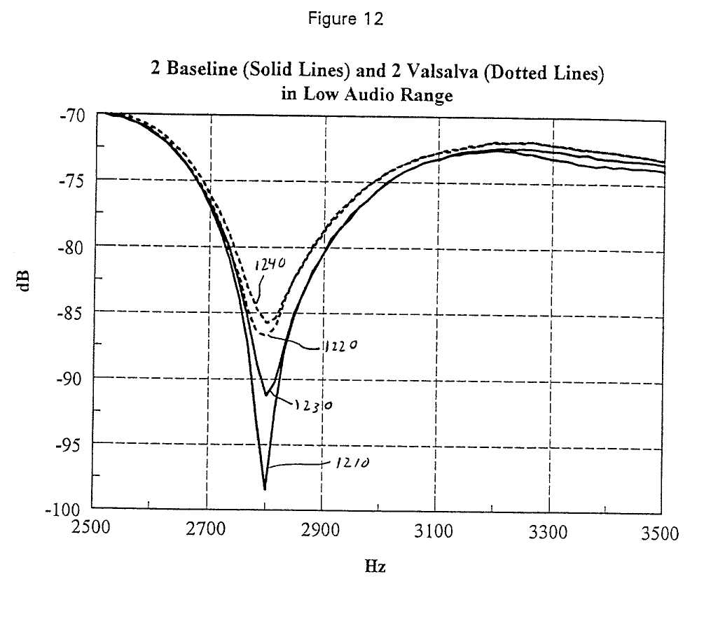

FIG. 12 shows plots 1210, 1220, 1230, 1240 of received signal strength obtained from a first subject, by utilizing the method and system of the present invention. These plots show that a frequency of about 2.8 kHz can be used to determine whether this brain is under pressure or not. This frequency corresponds to a brain/skull continuum frequency, and can be used along with other frequencies (such as the brain resonance frequency at a higher frequency value) to accurately determine CSF pressure.

A first baseline at 2.8 kHz, shown by the curve 1210 in FIG. 12, gives a received signal strength of about −98 dB. A valsalva was then performed to simulate a “brain pressure” event, where this valsalva occurred about six seconds after the first baseline. A valsalva is when someone holds their breath and pushes hard, thereby causing some CSF pressure increase while blood flow to the brain is partially blocked off. It has been estimated that a typical valsalva provides about a 20 mm of water pressure increase. During the first valsalva, the measured signal strength at 2.8 kHz was about −87 dB, as shown by the curve 1220, which is an 11 dB increase in signal strength due to the high pressure event in the head. The subject then stopped the valsalva and a measurement at a second baseline (non-pressure condition) was measured about six seconds later. The received signal at the second baseline was about −92 dB, as shown by the curve 1230, which is an increase from the first baseline. This is due to the brain trying to compensate for the previous high-pressure event (valsalva) that occurred not too long ago. In effect, the brain is accommodating for the high pressure event to try to make it less of a problem. A second valsalva was performed after the second baseline, and a signal measurement of about −86 dB was obtained, as shown by the curve 1240. Again, this measurement is greater than the previous baseline event, thereby providing an indication of an amount of pressure in the brain. The time order of these events is: first baseline, first valsalva, second baseline, second valsalva, third baseline, and third valsalva.

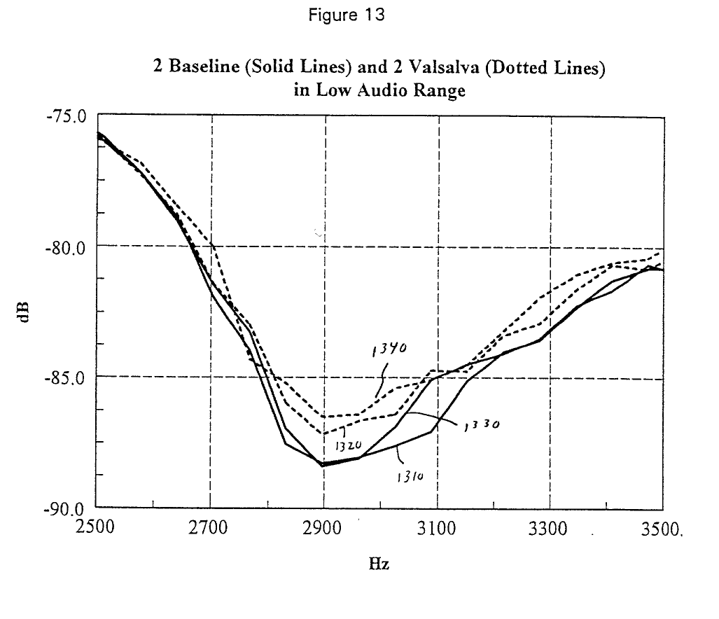

FIG. 13 shows plots 1310, 1320, 1330, 1340 of received signal strength obtained from a second subject, by utilizing the method and system of the present invention. These plots also show a useful resonant frequency value of around 2.9 kHz for this subject, whereby differences in no-pressure and pressure events can be determined by a received signal strength due to a 2.9 KHz pulse applied to the subject's head.

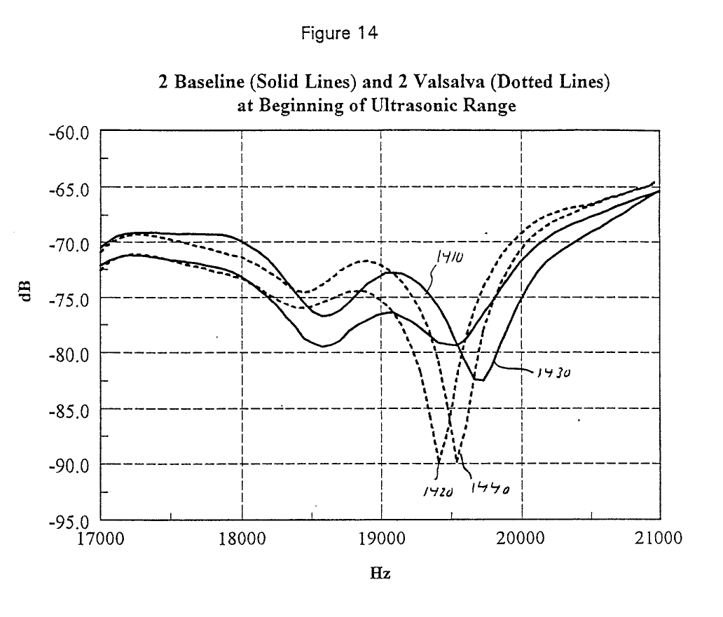

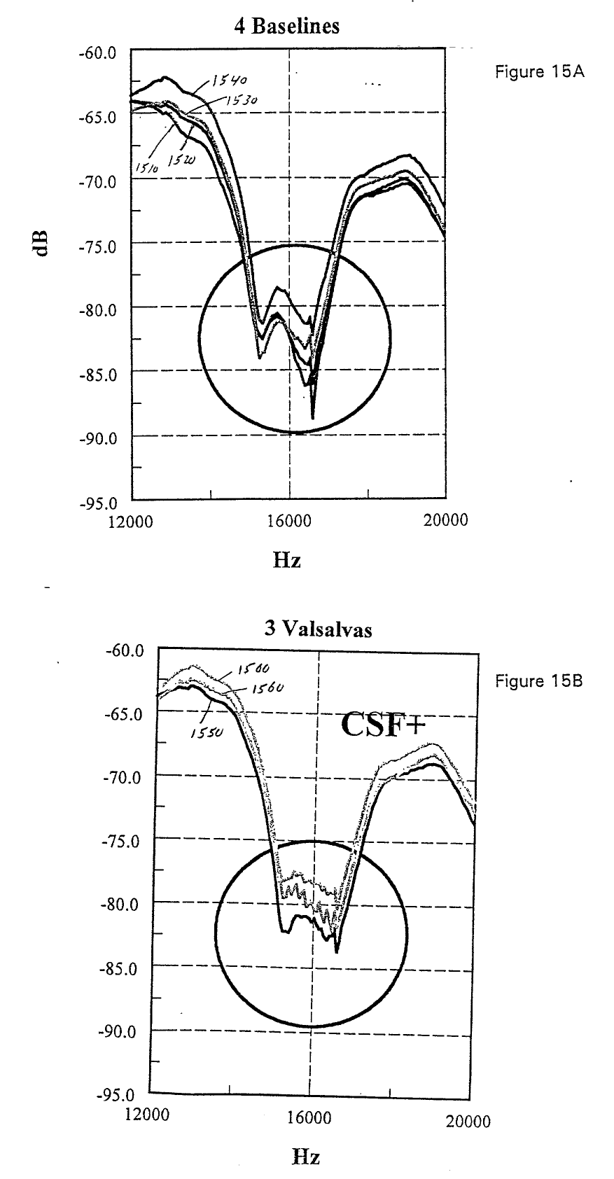

FIG. 14 is shows plots 1410, 1420, 1430, 1440 of received signal strength obtained from a subject at a high audio frequency range, by providing a signal to the subject's head and by sweeping the frequency of that signal over a short period of time (e.g., sweep from 2 kHz to 36 kHz over a period of 10 milliseconds). The data from these plots shows a useful brain resonant frequency of about 16.5 kHz, which can be used to determine if a brain is not under pressure (received signal strength of between −89 dB to −85 dB) or if the brain is under pressure (received signal strength of between −80 dB and −78 dB).

FIG. 15A shows plots 1510, 1520, 1530, 1540 of baselines from four different subjects that were obtained using a high-audio range applied to the respective heads of the four different subjects. FIG. 15B shows plots 1550, 1560, 1570 of high-pressure events (valsalvas) for three of the four different subjects using the same signal. These plots confirm that measuring a signal strength at an appropriate brain resonant frequency (or harmonic thereof) provides an indication as to whether or not a brain is experiencing increased CSF pressure, and if so, the rate at which that increase is occurring.

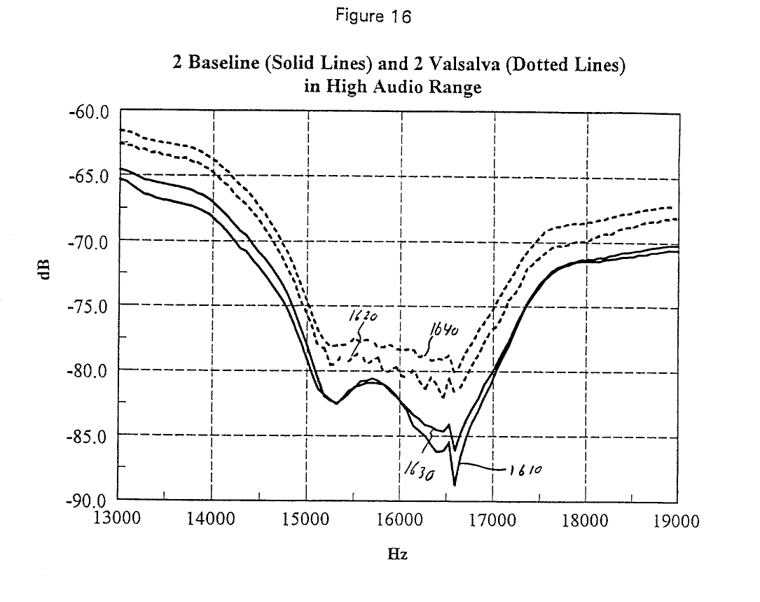

FIG. 16 shows plots 1610, 1620, 1630, 1640 of received signal strength for a subject at the start of the ultrasound frequency range for a signal provided to the subject's head. These plots show that at around 19.5 kHz, which is a harmonic of the brain resonant frequency, a difference between received signal strengths due to brain under-pressure/not-under-pressure exists. Note that, unlike the lower frequency signals discussed above, at this higher frequency, a “flipping over” effect is seen, in that the brain-under-pressure event results in a lower signal strength than the brain-not-under-pressure event. In any event, it is the relative difference that is being measured in the present invention to determine whether a CSF high pressure event is occurring, and to get an estimate of how much pressure that it is.

It is believed that this reversal may be due to the dominance of the skull resonance in the total coupled resonance at the lower audio frequencies.

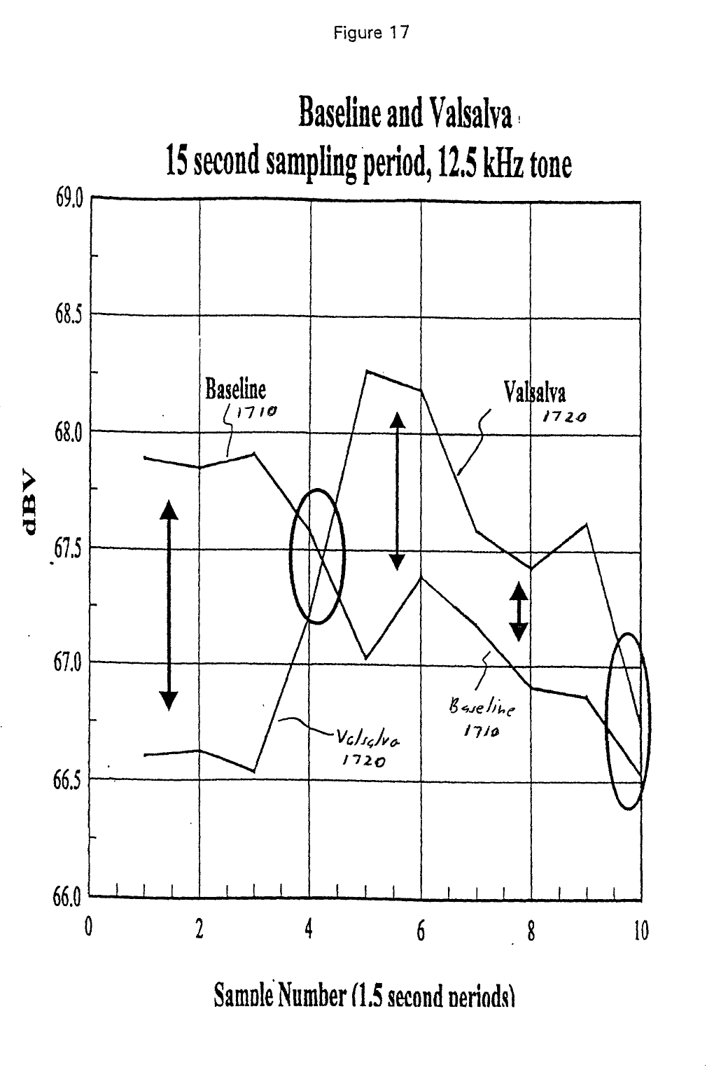

FIG. 17 shows plots of baseline (plot 1710) and valsalva (plot 1720) received signal strength (due to 12.5 kHz signals transmitted to the subject's head) for signals received over a 15 second period, whereby samples were taken every 1.5 seconds during the 15 second period. These plots verify that there is a difference in received signal strength due to a high-pressure event (valsalva) and a non-high-pressure event. While the difference in amplitude between the two received signal strengths “flips over” as time progresses, it is the difference itself that can be detected to determine the occurrence of such a high pressure event in the head of a subject.

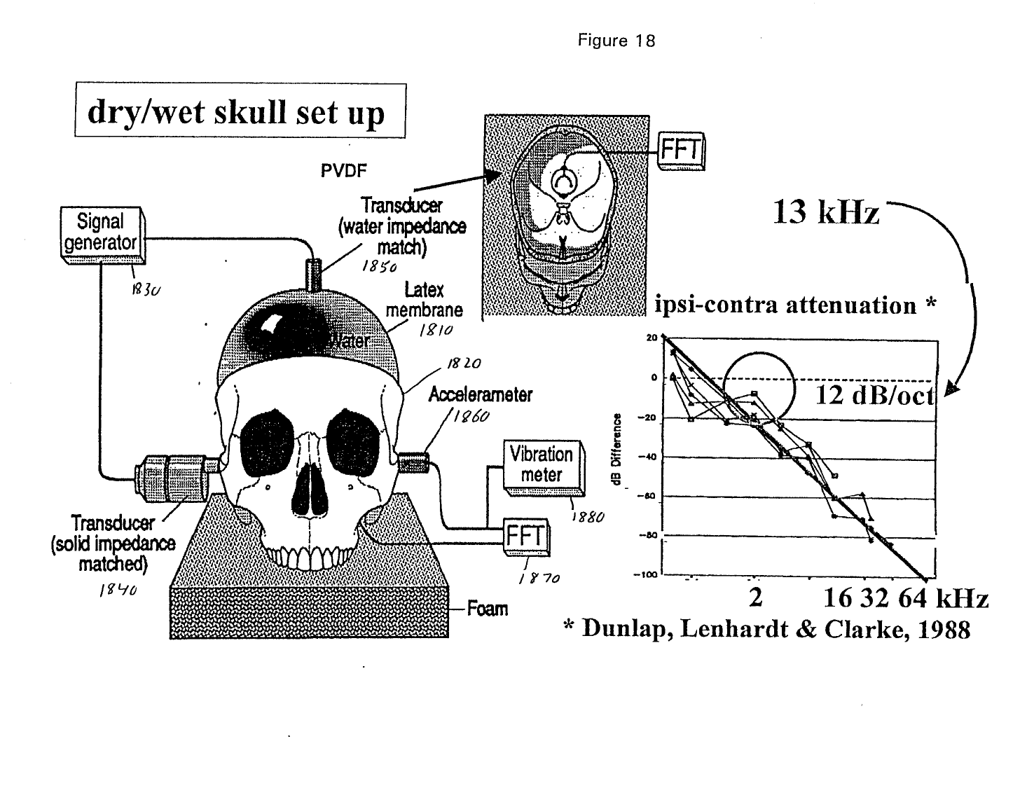

FIG. 18 shows a setup to test the concept of the present invention, in which a water balloon 1810 was placed inside an actual skull 1820, and signals were provided to the skull (by way of a signal generator 1830, a first transducer 1840 and a second transducer 1850) and signals were measured from an opposite location of the skull (by way of accelerometer 1860, FFT analyzer 1870 and vibration meter 1880). The plots provided in FIG. 18 show attenuation over a frequency range of about 1 kHz to 64 kHz in an empty skull.

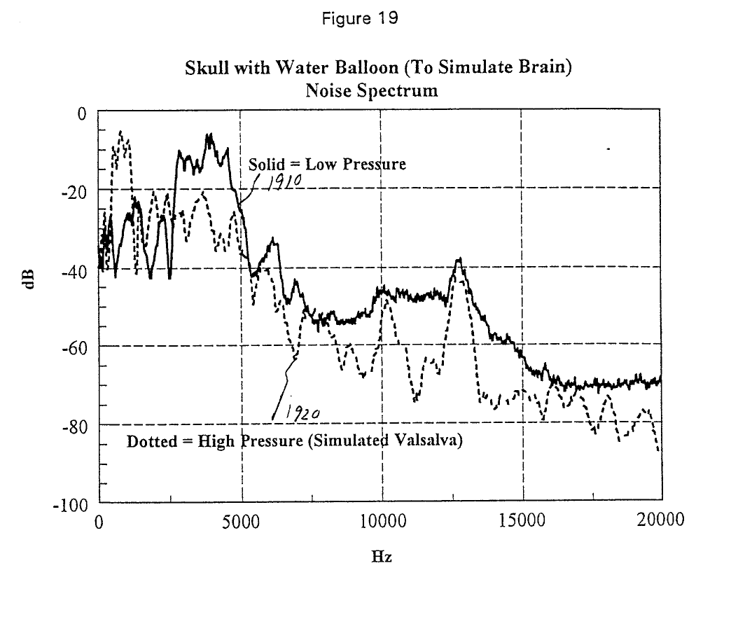

Based on the setup shown in FIG. 18, results were obtained from a non-pressure condition and from a pressure condition (water balloon was pushed to simulate the high-pressure event). At frequencies of about 2.5 kHz and higher, the high pressure event resulted in a lower received signal strength. Also, a brain resonance at about 12.5 kHz can be seen in FIG. 18. FIG. 19 shows plots 1910, 1920 of received signal strength over a range of frequencies for a low-pressure condition (plot 1910) and a high-pressure condition (plot 1920) using the setup of FIG. 18. As can be seen in FIG. 19, there is a difference in the received signal strength based on whether or not the brain is under pressure, and this difference can be detected in the present invention by the use of transducers and sensors in a non-invasive manner to determine the amount, if any, of CSF pressure in the skull. FIG. 19 also shows that the difference in the received signal strength is more prominent at the brain resonance frequency and harmonics thereof.

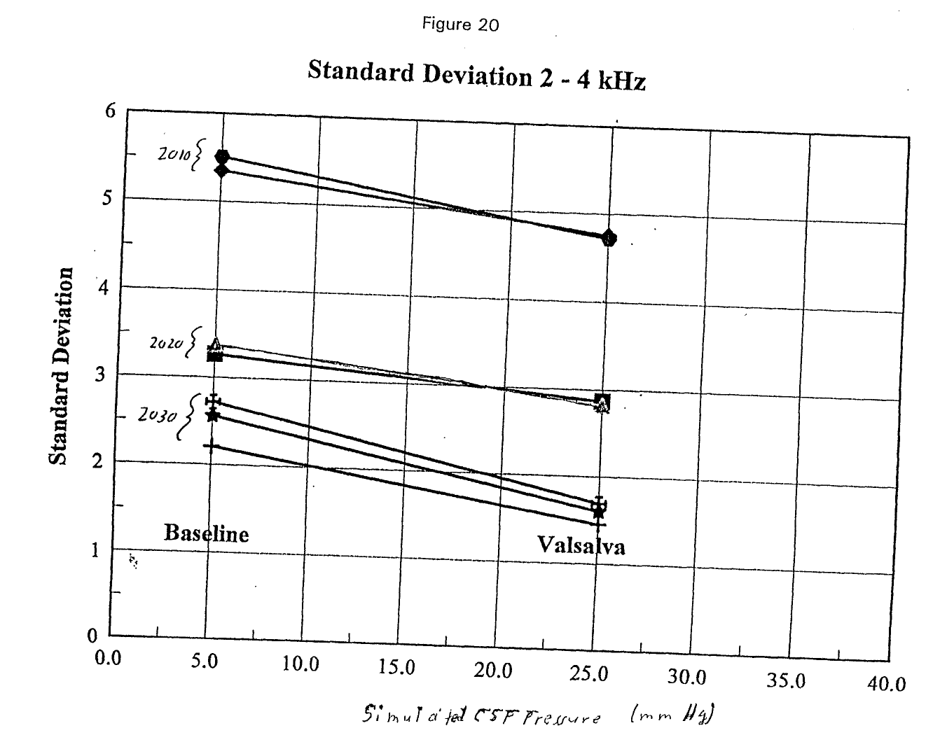

A third embodiment of the present invention will be described below. FIG. 20 shows plots 2010, 2020, 2030 of standard deviation obtained for three different subjects, in which the baseline standard deviation is higher than the valsalva standard deviation. The standard deviation values were obtained over a range of frequencies (audio up to lower ultrasonic frequency range) by sweeping an input signal to the head over a range of frequencies. As discussed above with respect to the first embodiment, the exact frequency of the brain resonance varies from person to person and also depends on the location at which the signals are provided to the brain. Also, the intensity of the received signal varies. However, as seen in FIG. 20, the degree of variability in a given frequency range tends to remain constant with constant CSF pressure, and varies reliably with increased pressure (standard deviation decreases).

The standard deviation is a statistical measure of the variability that does not depend on the frequency or intensity of the resonances; it is computed from all of the points between 2 kHz and 4 kHz in the example shown in FIG. 20 (a larger range of from 2 kHz to 36 kHz or some subset thereof would be more preferable). The plots of FIG. 20 show a consistent difference in standard deviation with changes in pressure. The slope of the plots 2010, 2020, 2030 in FIG. 20 measures this change, with the slope being practically the same for the three subjects. By projecting a line along the slope, CSF pressure can readily be extracted from a change in standard deviation.

In the third embodiment, a signal is swept across a range of frequencies and is provided to a head of a patient. The use of a swept tone signal is to compensate for the fact that the changes in dB with CSF pressure are not very consistent at any one frequency, and so the third embodiment takes measurements in a range of tones to compute a reliable CSF pressure. The sweep rate may be in the order of milliseconds (e.g., sweep of a signal from 2 kHz to 30 kHz in 10 milliseconds), but any comparable sweep rate and sweep time may be utilized while remaining within the scope of the invention.

Thus, for example, assume that a standard deviation of 4.0 was obtained from a patient (when his brain was under normal pressure, say 5 mm HG), and then a standard deviation of 3.8 was obtained (which would correspond to a pressure of 10 mm HG, for example), and then a standard deviation of 3.6 was obtained (which would corresponds to a pressure of 15 mm HG, for example). With this data, if a standard deviation of 3.4 was then obtained from the patient using a swept frequency signal, this would correspond to an estimated CSF pressure of 20 mm HG, using the system and method of the third embodiment.

While preferred embodiments of the present invention have been described in detail, modifications of those embodiments may be made by one skilled in the art, without departing from the spirit and scope of the present invention as described above and as exemplified in the appended claims.

What is claimed is:

1. A method of estimating an intracranial pressure within a head of a subject, comprising:
providing a signal to the head of the subject;
measuring an amplitude of a received signal at a location on the head of the subject different from where the signal was provided to the head of the subject; and
determining the intracranial pressure based on the amplitude of the received signal.

2. The method according to claim 1, wherein the signal is swept over a range of frequencies from 2 kHz to 36 kHz, and wherein the amplitude of the received signal is obtained over the range of frequencies in order to obtain an average amplitude value that is used to determine the intercranial pressure.

3. The method according to claim 1, wherein the signal is provided to the head of the subject at different positions on the head, and
wherein the amplitude of the received signal is measured by sensors provided at positions opposite to the positions in which the signal was provided to the head of the subject.

4. The method according to claim 1, further comprising:
providing a second signal to the head of the subject;
measuring an amplitude of a second received signal at a location on the head of the subject different from where the second signal was provided to the head of the subject; and
determining a change in the intracranial pressure based on a difference between the amplitude of the received signal and the amplitude of the second received signal.

5. A method of estimating an intracranial pressure within a head of a subject, comprising:
providing a signal to the head of the subject at at least two different times and at at least two different frequency values, respectively, that correspond to a brain resonant frequency and a harmonic of the brain resonant frequency;
measuring an amplitude of a received signal at a location on the head of the subject different from where the signal was provided to the head of the subject, the measuring being performed at the first and second times; and
determining the intercranial pressure based on the amplitude of the received signal at the first and second times.

6. The method according to claim 5, wherein the signal is provided at the first time at a value of between 10 kHz and 15 kHz, and
wherein the signal is provided at the second time at a value greater than the value used at the first time.

7. The method according to claim 5, wherein the signal is provided at the first time at a frequency of between 10 kHz and 15 kHz, and
wherein the signal is provided at the second time at a frequency less than the frequency used at the first time.

8. The method according to claim 8, further comprising:
providing a second signal to the head of the subject;
measuring an amplitude of a second received signal at a location on the head of the subject different from where the second signal was provided to the head of the subject; and
determining a change in the intracranial pressure based on a difference between the amplitude of the received signal and the amplitude of the second received signal.

9. An apparatus for estimating an intracranial pressure within a head of a subject, comprising:
an adjustable headband that includes at least one transducer and one sensor, the at least one transducer providing a signal to the head of the subject; and
a computation unit that estimates the intracranial pressure and that provides a signal to the at least one transducer to output the signal,
wherein an amplitude of a received signal is measured by the at least one sensor at a location on the head of the subject different from where the signal was provided to the head of the subject, and
wherein the intracranial pressure is estimated based on the amplitude of the received signal as provided to the computation unit by the at least one sensor.

10. The apparatus according to claim 9, wherein the signal is swept over a range of frequencies in an audio band and in an ultrasonic band, and
wherein the computation unit estimates the intercranial pressure based on a standard deviation value obtained based on the amplitude of the received signal obtained over the range of frequencies.
